(19) United States
(12) Patent Application Publication
(10) Pub. No.: US 2011/0129880 A1
(43) Pub. Date: Jun. 2, 2011

(54) PROCESS FOR TREATING BIOMASS TO INCREASE ACCESSIBILITY OF POLYSACCARIDES CONTAINED THEREIN TO HYDROLYSIS AND SUBSEQUENT FERMENTATION, AND POLYSACCHARIDES WITH INCREASED ACCESSIBILITY

(75) Inventors: Herbert T. Conners, Landenberg, PA (US); Patrick J. Cowan, Hockessin, DE (US); John C. Gast, Hockessin, DE (US); Robert P. O'Flynn O'Brien, Hockessin, DE (US)

(73) Assignee: Hercules Incorporated, Wilmington, DE (US)

(21) Appl. No.: 12/917,836

(22) Filed: Nov. 2, 2010

Related U.S. Application Data
(60) Provisional application No. 61/257,302, filed on Nov. 2, 2009.
(60) Provisional application No. 61/257,306, filed on Nov. 2, 2009.

(51) Int. Cl. C12P 19/04; C12P 7/02; C12P 7/16; C12P 7/10; C07H 1/00

(52) U.S. Cl. 435/101; 435/155; 435/160; 435/165; 536/123.1

(57) ABSTRACT

In this invention, a process for producing fermentable sugars derivable from biomass that contains polysaccharide, such as cellulose, which has been made increasingly accessible as a substrate for enzymatic degradation or other methods of depolymerization. The process of the present invention increases accessibility of polysaccharides, typically present in biomass and produces polysaccharides with increased accessibility. The polysaccharides with increased accessibility may be subsequently saccharified to yield fermentable sugars. These fermentable sugars are subsequently able to be fermented to produce various target chemicals, such as alcohols, aldehydes, ketones or acids.

### RELATED APPLICATIONS

This application claims the benefit of U.S. Provisional Application Ser. No. 61/257,302, and U.S. Provisional Application Ser. No. 61/257,306, the disclosures of which are incorporated herein by reference in their entireties.

### FIELD OF THE INVENTION

This invention relates to polysaccharides, particularly to cellulose, and to a process for converting polysaccharide to sugars which can be subsequently fermented.

### BACKGROUND OF THE INVENTION

Polysaccharides contain structured and even crystalline portions which make them less soluble in water and also difficult to break down to their recurring units to obtain the underlying monomeric units. In the case of cellulose, these monomeric units are glucose units which can be converted to useful compounds, including ethanol or other target molecules obtained through fermentation.

Ethanol and other chemical fermentation products typically have been produced from sugars derived from high value feedstocks which are typically high in starches and sugars, such as corn. These high value feedstocks also have high value as food or feed.

It has long been a goal of chemical researchers to improve the efficiency of depolymerizing polysaccharides to obtain monomeric and/or oligomeric sugar units that make up the polysaccharide repeating units. It is desirable to increase the rate of reaction to yield free monomer and/or oligomers units in order to increase the amount of alcohol or other target molecules that may be obtained by fermentation of the monomeric and/or oligomeric units.

Much research effort has been directed toward enzymes for depolymerizing polysaccharides, especially to obtain fermentable sugars which can be converted by fermentation to target chemicals such as alcohols.

However, some polysaccharides, such as cellulose, are relatively resistant to depolymerization due to their rigid, tightly bound crystalline chains. Thus the rate of hydrolysis reaction to yield monomer may be insufficient for efficient use of these polysaccharides in general, and cellulose in particular, as a source for saccharide monomers in commercial processes. Enzymatic hydrolysis and fermentation in particular can also take much longer for such polysaccharides. This in turn adversely affects the yield and the cost of fermentation products produced using such polysaccharides as substrates.

A number of methods have been developed to disrupt the ordered regions of polysaccharides to obtain more efficient monomer release. Most of these methods involve pre-treatment of the polysaccharide. Pretreatments chemically and/or physically help to overcome resistance to enzymatic hydrolysis for cellulose and are used to enhance cellulase action. Physical pretreatments for plant lignocellulosics include size reduction, steam explosion, irradiation, cryomilling, and freeze explosion. Chemical pretreatments include dilute acid hydrolysis, buffered solvent pumping, alkali or alkali/H₂O₂ delignification, solvents, ammonia, and microbial or enzymatic methods.

These methods include acid hydrolysis, described in U.S. Pat. No. 5,916,780 to Foody, et al. The referenced patent also describes the deficiency of acid hydrolysis and teaches use of pretreatment and treatments by enzymatic hydrolysis.

U.S. Pat. No. 5,846,787 to Ladisch, et al. describes enzymatically hydrolyzing a pretreated cellulosic material in the presence of a cellulase enzyme where the pretreatment consists of heating the cellulosic material in water.

In US Patent Application No. 20070031918 A1, a biomass is pretreated using a low concentration of aqueous ammonia at high biomass concentration. The pretreated biomass is further hydrolyzed with saccharification enzymes wherein fermentable sugars released by saccharification may be utilized for the production of target chemicals by fermentation.

Zhao, et. al. (Zhao, Y. Wang, Y, Zhu, J. Y., Ragauskas, A., Deng, Y. in Biotechnology and Bioengineering (2008) 99(6) 1320-1328) have shown that high levels of urea, when combined with sodium hydroxide as a means of swelling the cellulosic matrix, improves the accessibility of the isolated cellulose for subsequent enzymatic hydrolysis. This may be attributed to the effect of the urea in disrupting the hydrogen bonding structures that are important in producing the more ordered regions of the polysaccharide.

J. Borsa, I. Tanczos and I. Rusznak, “Acid Hydrolysis of Carboxymethylcellulose of Low Degree of Substitution”, Colloid & Polymer Science, 268:649-657 (1990)) has shown that introduction of very low levels of carboxymethylation accelerates the initial rate of hydrolysis when cellulose is subjected to acid hydrolysis.

The Brosa process treats cotton fabrics by dipping in caustic and then sodium chloroacetate solution resulting in mild surface substitution at levels below 0.1 D.S. In FIG. 1, a maximum D.S. of about 95 millimoles per basemole after 20 minutes of carboxymethylation, or 0.095 D.S using the numbering for D.S. of carboxymethyl groups per anhydroglucose unit is shown.

Borsa et al. used a large excess of sodium hydroxide (of mercerizing strength) but a small amount of chloroacetic acid. Further, reported yields in Borsa, et al. of hydrolyzate are on the order of 0 to 35 milligrams per gram, or not more than 3.5% while the untreated cotton control yields about 2.5% hydrolysis under the same conditions.

In U.S. Pat. No. 6,602,994 to Cash, et al., it has been shown that low levels of cellulosic derivatization aids in reducing the amount of mechanical energy required for defibrillation. Cellulose is first swelled with alkali and then reacted with chloroacetic acid or other suitable reagents to obtain derivatized cellulose.

In U.S. patent application Ser. No. 12/669,584 filed on Feb. 3, 2010, a process for producing fermentable sugars derivable from biomass comprising the step of treating the biomass with a swelling agent and contacting the biomass with a derivatization agent to produce a derivatized polysaccharide with increased accessibility was taught. Polysaccharide contained in the biomass was derivatized by addition of a derivatization agent that reacts with the hydroxyl, carboxyl, or other functional groups of the polysaccharide. The derivatized polysaccharide with increased accessibility may be used as a substrate for enzymatic hydrolysis or other methods of depolymerization, and so that the derivatized polysacharride remains substantially insoluble in the medium conducive to enzymatic hydrolysis or other methods of depolymerization. The derivatized polysaccharide with increased accessibility produced by the above mentioned process can be treated with a saccharification enzyme or enzymes, such as cellulase enzyme, under suitable conditions to saccharify the derivatized polysaccharide and produce fermentable sugars.

### SUMMARY OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In this invention, a process is described that makes biomass that contains polysaccharide, such as cellulose, increasingly accessible as a substrate for enzymatic degradation or other methods of depolymerization.

One aspect of the present invention relates to a process for producing fermentable sugars derivable from biomass that contains polysaccharide. The process comprises the steps of obtaining a biomass that contains polysaccharide; treating the biomass with a swelling agent; contacting the biomass with a disrupting agent to produce a polysaccharide with increased accessibility. The polysaccharide with increased accessibility is converted to fermentable sugars by hydrolysis, such as through the use of one or more saccharification enzymes.

The polysaccharide with increased accessibility exhibits increased conversion to soluble components when subjected to a relevant Enzyme Accessibility Test, when compared to polysaccharide obtained from the biomass containing polysaccharide, which has been treated with the swelling agent but has not been contacted with the disrupting agent.

Another aspect of the present invention is a process for converting polysaccharide into fermentable sugars, which can then be treated with at least one biocatalyst able to ferment the sugars, to produce a target chemical under suitable fermentation conditions. The conversion process comprising the steps of obtaining a biomass containing polysaccharide and treating the biomass in a media with a swelling agent. The polysaccharide contained in the biomass is then disrupted by addition of a disrupting agent that incorporates within the polysaccharide and the disrupting agent is retained within the polysaccharide matrix upon removal or neutralization of the swelling agent, with the result that the disrupted polysaccharide exhibits increased accessibility.

While not wishing to be bound by theory, a “polysaccharide with increased accessibility” is a polysaccharide in which the ordered structure of the polysaccharide is rendered less ordered by incorporation within the matrix of the polysaccharide molecular structure, disrupting agents that interrupt the ability of the polysaccharide to return to an ordered structure upon removal or neutralization of the swelling agent from the polysaccharide. Reduction of order in the polysaccharide is obtained without substantially altering the molecular order of the polysaccharide, that is, without substantially altering the anhydro-ring structure that is inherent to the polysaccharide molecular structure. Examples of polysaccharide with increased accessibility from this process include instances where the disrupting agent is substantive to the polysaccharide and remains associated with the polysaccharide, even after removal or neutralization of the disrupting agent.

In a one aspect of the invention, the polysaccharide in the biomass is contacted with a swelling agent having sufficient alkalinity to swell the polysaccharide. Alkalinity can be provided by treatment with an alkaline solution or vapor with sufficient alkalinity to swell the polysaccharide. The swelling agent may be present in a media wherein the media in which the swelling agent is contained may be in liquid form and may be any alkaline solution comprising water, water miscible solvents such as alcohol or acetone and water/water miscible solvent mixtures. If the media in which the swelling agent is contained is in a vapor form, it may comprise either air or other readily obtainable or generated gas.

While not wishing to be bound by theory, in another aspect of the invention, the polysaccharide is disrupted by addition of a disrupting agent that incorporates within the biomass containing polysaccharide with the polysaccharide exhibiting increased accessibility. The swelling agent may be removed from the biomass containing polysaccharide or neutralized prior to subsequent conversion to fermentable sugars in order not to inhibit or interfere with effectiveness of the one or more saccharification enzymes used to produce the fermentable sugars from the polysaccharide.

In another aspect of the invention, the disrupting agent is a material that incorporates within biomass containing polysaccharide through diffusion into the polysaccharide.

In yet another aspect of the invention, an effective amount of the disrupting agent is retained within biomass that contains polysaccharide upon removal or neutralization of the swelling agent by being substantive to or entrapped within the polysaccharide matrix.

Particularly useful disrupting agents are those that are substantive to the polysaccharide, showing preferential adsorption onto the polysaccharide. Particularly useful substantive disrupting agents remain associated with the polysaccharide upon removal or neutralization of the swelling agent from the biomass.

Disrupting agents that effectively disrupt the polysaccharide following incorporation into the polysaccharide and retention following removal of the swelling agent include, but are not limited to, small molecules that physically adsorb onto or are substantive to the polysaccharide or those that become entrapped in the polysaccharide matrix. The disrupting agents of use in the present invention have a molecular weight between about 60 to about 400 Daltons. These molecules include oligomers or monomers of similar materials to the polysaccharide or fermentable sugars obtained from the polysaccharide, such as glucose, maltose or dextrose. The preferred disrupting agent may be selected from the group consisting of fermentable sugars, nonfermentable sugars, hydroxyl or lactone containing molecules derived from sugar degradation, urea, amines, and polyols. The disrupting agent may selected from the group consisting of organic molecules containing hydroxyl groups, lactones, and water soluble ethers. The disrupting agent may also be selected from the group consisting of amines, amino acids, sulfates, and phosphates. Hydroxyl or lactone containing molecules derived from sugar degradation, polyols, ethers, furans, and related hydrophilic compounds may be incorporated into the ordered structure to give similar disruption, and a related reduction of order. Products of subsequent fermentation such as ethanol, 1,3 propanediol, propylene glycol, glycerol, propanol, butanol, etc. may also be used as a disrupting agent. Mixtures of the above may also be used.

In another aspect of the invention, the polysaccharide containing the disrupting agent is then treated to remove or neutralize the swelling agent. Various methods are available for removing or neutralizing the swelling agent. In a specific example, an alkaline swelling agent is pH adjusted to a level suitable for a subsequent conversion of the polysaccharide with increased accessibility to monomer or oligomer units by enzymatic hydrolysis. The polysaccharide with increased accessibility is converted to monomeric and/or oligomeric sugar units by enzymatic hydrolysis, and these available monomeric and/or oligomeric sugar units may now be converted into various desirable target chemicals by fermentation or other chemical processes, such as acid hydrolysis.

The polysaccharide with increased accessibility produced by the above mentioned process can be treated with a saccharification enzyme or enzymes, such as cellulase enzyme, under suitable conditions to produce fermentable sugars. This hydrolytic degradation depolymerizes the disrupted polysaccharide making the monomeric and oligomeric units which comprise the fermentable sugars available for a number of uses, including production of target chemicals by fermentation.

In a further aspect of the invention, the products arising from hydrolysis of the disrupted polysaccharide, which contain the monomeric and oligomeric units, is then treated with a yeast or related organism or enzyme under suitable fermentation conditions to induce enzymatic degradation of the monomeric and/or oligomeric units such as fermentation. Fermentation breaks bonds in the sugar rings and results in the monomer or oligomer units being converted to target chemicals. The target chemicals obtained from the above described process may be selected from the group consisting of alcohols, aldehydes, ketones and acids. The alcohols produced by the above described process may include the group consisting of methanol, ethanol, propanol, 1,2 propanediol, glycerol, and butanol. The preferred alcohol being ethanol.

### DETAILED DESCRIPTION OF THE INVENTION

One aspect of this invention relates to a process that makes a biomass that contains polysaccharide, such as cellulose, increasingly accessible as a substrate for enzymatic degradation or other methods of depolymerization. In one example, this is achieved by forming a polysaccharide with increased accessibility following treatment with a swelling agent and a disrupting agent that incorporates and retains within the polysaccharide matrix following removal or neutralization of the swelling agent. The polysaccharide exhibits increased accessibility upon incorporation of the disrupting agent within the matrix of the polysaccharide molecular structure.

Another aspect of this invention relates to a process for preparation of target chemicals from polysaccharide substrates with increased accessibility in which said processes comprises, in combination or sequence, hydrolysis of the polysaccharide substrates with increased accessibility to fermentable sugars and enzymatic degradation of such fermentable sugars such as occurs in fermentation or other chemical processes.

In this disclosure, a number of terms are used. The following definitions are provided.

The term “fermentation” refers to an enzymatic process whereby conversion of a fermentable material to smaller molecules along with CO₂ and water occurs.

The term “fermentable sugar” refers to oligosaccharides, monosaccharides, and other small molecules derived from polysaccharides that can be used as a carbon source by a microorganism, or an enzyme, in a fermentation process.

The term “lignocellulosic” refers to a composition or biomass comprising both lignin and cellulose. Lignocellulosic material may also comprise hemicellulose.

The term “cellulosic” refers to a composition comprising cellulose.

The term “disrupting agent” refers to a material that when incorporated and retained within the matrix of an ordered polysaccharide material renders the ordered polysaccharide material less ordered and more accessible to enzyme degradation.

The term “target chemical” refers to a chemical produced by fermentation or chemical alteration from a polysaccharide exhibiting increased accessibility rendered to be more accessible by the process of the invention.

The term “saccharification” refers to the production of fermentable sugars from polysaccharides.

The phrase “suitable conditions to produce fermentable sugars” refers to conditions such as pH, composition of medium, and temperature under which saccharification enzymes are active.

The term “degree of substitution” (D.S.) means the average number of hydroxyl groups, per monomer unit in the polysaccharide molecule which have been substituted. For example in cellulose, if on average only one of the positions on each anhydroglucose unit are substituted, the D.S. is designated as 1, if on average two of the positions on each anhydroglucose unit are reacted, the D.S. is designated as 2. The highest available D.S. for cellulose is 3, which means each hydroxyl unit of the anhydroglucose unit is substituted.

The term “molar substitution” (M.S.) refers to the average number of moles of substituent groups per monomer unit of the polysaccharide.

The term “polysaccharide with increased accessibility” refers to polysaccharides exhibiting increased accessibility to enzyme as determined using a relevant Enzyme Accessibility Test.

The term “biomass” refers to material containing polysaccharide such as any cellulosic or lignocellulosic materials and includes materials comprising polysaccharides, such as cellulose, and optionally further comprising hemicellulose, lignin, starch, oligosaccharides and/or monosaccharides. Biomass may also comprise additional components, such as protein and/or lipid. According to the invention, biomass may be derived from a single source, or biomass can comprise a mixture derived from more than one source; for example, biomass could comprise a mixture of corn cobs and corn stover, or a mixture of grass and leaves. Biomass or materials that contain substantial amounts of biomass includes, but are not limited to, bioenergy crops, agricultural residues, municipal solid waste, industrial solid waste, sludge from paper manufacture, paper and paperboard, yard waste, wood and forestry waste. Examples of biomass include, but are not limited to, corn grain, corn cobs, crop residues such as corn husks, corn stover, grasses, wheat, wheat straw, barley, barley straw, hay, rice straw, cotton, cotton linters, switchgrass, waste paper or post consumer paper, sugar cane bagasse, sorghum, soy, components obtained from milling of grains, trees, branches, roots, leaves, wood chips, sawdust, shrubs and bushes, vegetables, fruits, flowers and animal manure. In one embodiment, biomass that is useful for the invention includes biomass that has a relatively high carbohydrate value, is relatively dense, and/or is relatively easy to collect, transport, store and/or handle. In one embodiment of the invention, biomass that is useful includes corn cobs, corn stover and sugar cane bagasse.

The biomass may also comprise various suitable polysaccharides which include, chitin, chitosan, guar gum, pectin, alginate, agar, xanthan, starch, amylose, amylopectin, alternan, gellan, mutan, dextran, pullulan, fructan, locust bean gum, carrageenan, glycogen, glycosaminoglycans, murein, bacterial capsular polysaccharides, and derivatives thereof. Mixtures of these polysaccharides may be employed. Preferred polysaccharides are cellulose, derivatized cellulose, chitin, chitosan, pectin, agar, starch, carrageenan, and derivatives thereof, used singly or in combination, with cellulose being most preferred. The cellulose may be obtained from any available source, including, by way of example only, chemical pulps, mechanical pulps, thermal mechanical pulps, chemical-thermal mechanical pulps, recycled fibers, newsprint, cotton, soybean hulls, pea hulls, corn hulls, flax, hemp, jute, ramie, kenaf, manila hemp, sisal hemp, bagasse, corn, wheat, bamboo, velonia, bacteria, algae and fungi. Other sources of cellulose include purified, optionally bleached wood pulps produced from sulfite, kraft, or prehydrolyzed kraft pulping processes; purified and non-purified cotton linters; fruits; and vegetables. Cellulose containing materials most often include lignin and are often referred to as lignocellulosics, which include the various wood, grass, and structural plant species found throughout the plant world, many of which are mentioned above.

Preferred derivatized celluloses include, but are not limited to, hydroxyethyl cellulose, ethylhydroxyethyl cellulose, carboxymethylcellulose, carboxymethylhydroxyethyl cellulose, hydroxypropylhydroxyethyl cellulose, methyl cellulose, ethylcellulose, methylhydroxypropyl cellulose, methylhydroxyethyl cellulose, carboxymethylmethyl cellulose, hydrophobically modified carboxymethylcellulose, hydrophobically modified hydroxyethyl cellulose, hydrophobically modified hydroxypropyl cellulose, hydrophobically modified ethylhydroxyethyl cellulose, hydrophobically modified carboxymethylhydroxyethyl cellulose, hydrophobically modified hydroxypropylhydroxyethyl cellulose, hydrophobically modified methyl cellulose, hydrophobically modified methylhydroxypropyl cellulose, hydrophobically modified methylhydroxyethyl cellulose, hydrophobically modified carboxymethylmethyl cellulose, nitrocellulose, cellulose acetate, cellulose sulfate, cellulose vinyl sulfate, cellulose phosphate, and cellulose phosphonate. Other polysaccharides may be similarly derivatized.

The biomass may be used directly as obtained from the source, or energy may be applied to the biomass to reduce the size, increase the exposed surface area, and/or increase the availability of polysaccharides present in the biomass to a swelling agent and to saccharification enzymes used in the second step of the method. Energy means useful for reducing the size, increasing the exposed surface area, and/or increasing the availability of cellulose, hemicellulose, and/or oligosaccharides present in the biomass to the swelling agent and to saccharification enzymes include, but are not limited to, milling, crushing, grinding, shredding, chopping, disc refining, ultrasound, thermomechanical and mechanical pulping, chemical pulping, and microwave.

Conditions for swelling polysaccharides should generally include, but are not limited to, treatment with an alkaline agent producing swelling of the polysaccharide. The swelling process is intended to make the polysaccharide more accessible to the placement or generation of the disrupting agent within the polysaccharide matrix. Swelling may be provided to various degrees and may involve treatment with one or more materials.

Alkaline conditions are preferably obtained by using alkali metal hydroxide. Any material that functions as an alkaline media for the polysaccharide of choice may be used as a swelling agent, and alternative swelling agents include alkali metal or alkaline earth metal oxides or hydroxides; alkali silicates; alkali aluminates; alkali carbonates; amines, including aliphatic hydrocarbon amines, especially tertiary amines; ammonia, ammonium hydroxide; tetramethyl ammonium hydroxide; lithium chloride; N-methyl morpholine N-oxide; and the like.

The concentration of the swelling agent can be at various levels though the results suggest that higher levels of swelling agent may produce more opportunity for incorporation of the disrupting agent. In particular if swelling agents such as those produced by the alkali metal hydroxides are used than concentrations that produce a significant degree of swelling, such as levels that produce relatively uniformly substituted cellulose derivatives, up to and including the so-called mercerization condition for cellulose, provide for opportunities for improved incorporation of the disrupting agent. The extent of swelling imparted by a particular swelling agent can depend on other conditions such as temperature. Variation of physical conditions that impact the extent of swelling are also included within the scope of this invention when the variation is used to increase the extent of disruption imparted by a disrupting agent incorporated into the polysaccharide using the varied condition.

The form of the swelling agent can also be of various types well known to those skillful in swelling polysaccharides. Most common are aqueous solutions of an alkaline material but also used are combinations of water and other solvents such as alcohols, acetone, or miscible solvents to form so-called slurries of swollen polysaccharides. Employing different types and ratios of cosolvents can produce various degrees of disorder in the final product after removal or neutralization of the swelling agent. Yet another common form of swelling agent would include penetrating gases such as ammonia which are capable of swelling polysaccharides under specific conditions.

Materials useful for disrupting the order of the polysaccharide can be of various types, as long as said disrupting agent can be substantive to, or entrapped within, the polysaccharide by a number of various processes. These disrupting agents are then retained in the polysaccharide matrix upon removal or neutralization of the swelling agent by a number of various processes, and which act to produce a product with increased accessibility for subsequent reactions or treatment with various materials. Combination of disrupting agents can also be used, including those that function by different mechanisms. Specific disrupting agents include, but are not limited to, materials such as sugars and oligiosaccharides such as glucose, maltose, or maltotriose that are substantive to the polysaccharide molecules. Of particular interest are disrupting agents which comprise fermentable sugars that are the resultant product from saccharification of the polysaccharide.

In certain cases, one may be able to utilize the fermentable sugars, which are the resultant product from saccharification of the polysaccharide, as the disrupting agent whereby a portion of the fermentable sugars which are the resultant product from saccharification of the polysaccharide is fed back in the process to contact the polysaccharide as a disrupting agent.

“Disruption” refers to any process whereby a disrupting agent becomes sufficiently associated or entrapped within or substantive to the polysaccharide, making the disrupted polysaccharide more accessible as a substrate for enzymatic degradation or other methods of depolymerization.

One particularly preferred method of producing the polysaccharide having increased accessibility pertains to the use of monomers or oligomers, the fermentable sugars, produced by the saccharification of the polysaccharide, fed back into the process to function as the disrupting agent for producing the polysaccharide having increased accessibility. There are a number of advantages of such a process. One being that by using a portion of the fermentable sugars produced as the disrupting agent avoids the introduction of other chemical species into the process that subsequently must be disposed of or neutralized. Additionally, while not wishing to be bound by theory, it is felt that the fermentable sugars are relatively compatible with the polysaccharide since they are obtained from the polysaccharide. For example, in the process for making cellulose with increased accessibility, glucose produced by hydrolysis of the cellulose, can be fed back in the process to function as the disrupting agent for the cellulose.

Isolation of the polysaccharide having increased accessibility involves removing or neutralization of the swelling agent by various means resulting in retention of the disrupting agent and partial or complete removal of the swelling agent.

A method of isolation is to remove or neutralize the swelling agent from the slurry containing the polysaccharide with increased accessibility, with a washing agent that is a poor or non-solvent to the disrupting agent. The conditions of the washing process as well as the composition of the washing agent may substantially impact the properties of the resulting disrupted polysaccharide. Among the washing process regimens that are of use in the present invention involve the use of water alone, water miscible solvents, such as alcohol or acetone, or water/water miscible solvent mixtures.

The polysaccharide with increased accessibility may be dried after the washing process. This may permit the storage of the polysaccharide with increased accessibility prior to its subsequent depolymerization to fermentable sugars. Alternatively, the polysaccharide with increased accessibility may be subsequently depolymerized by hydrolysis to fermentable sugars without being dried. This is a preferred process since the increased accessibility of the polysaccharide appears to be retained with an improvement in the yield of the fermentable sugars from the never dried polysaccharide with increased accessibility.

The polysaccharides with increased accessibility of this invention are subsequently depolymerized by hydrolysis under suitable conditions to produce fermentable sugars. Hydrolysis of the disrupted polysaccharide can be accomplished by treatment with acids, bases, steam or other thermal means, or enzymatically. Preferred methods of hydrolysis include treatment with enzymes, acids, or steam, with enzymatic hydrolysis being most preferred.

The fermentable sugars obtained by the above described process are then converted to target chemicals by enzymatic degradation such as occurs in fermentation.

One fermentation procedure consists simply of contacting the fermentable sugars under suitable fermentation conditions with yeast or related organisms or enzymes. Yeast contains enzymes which use fermentable sugars, such as glucose, to produce ethanol, water, and carbon dioxide as byproducts of the fermentation procedure. The carbon dioxide is released as a gas. The ethanol remains in the aqueous reaction media and can be removed and collected by any known procedure, such as distillation and purification, extraction, or membrane filtration. Other useful target chemicals may be likewise produced by fermentation.

### Enzyme Accessibility Test

In order to determine the degree of increased accessibility of a polysaccharide treated using the present process to enzymatic hydrolysis, when compared to a control polysaccharide, an Enzyme Accessibility Test is performed. Any statistically significant increase in the soluble portion of initial solids of the polysaccharide, when compared to an appropriate control, as determined by the following test, shall be considered to be indicative of a polysaccharide with increased accessibility. Please note, that the below-listed Enzyme Accessibility Test is relevant for determining increased accessibility of cellulose since it recites the use of cellulase and since the polysaccharide being tested is cellulose. An appropriate enzyme should be selected for the particular polysaccharide being tested in an Enzyme Accessibility Test for it to be considered relevant. Amounts of material used may also be modified when testing different polysaccharides.

The below-listed amounts of samples and reagents may be varied to account for weighing accuracy and availability of materials.

The following is an example of an Enzyme Accessibility Test which is relavent to cellulase accessibility of cellulose samples:

In 100 ml jars are added in order:

0.61 g Cellulase Enzyme (573 units*) Sigma EC 3.2.1.4 from Pennicillum funiculosum L#58H3291.

*1 unit=1 micromole of glucose from cellulose in 1 hour at pH 5 at 37° C. (as defined by Sigma-Aldrich for the enzyme used).

3.00 g cellulosic furnish (dry basis) such as cotton linters, wood pulp or biomass.

75.00 g Sodium Phosphate buffer adjusted to pH 5.00, 50 milliMolar buffer. This buffer solution may be made by mixing 50 milliMolar monobasic and dibasic sodium phosphate buffers.

(J. T. Baker Analyzed ACS Reagent grade, CAS #07558-79-4 and CAS #10049-21-5).

The jars are capped and shaken repeatedly over 5 minutes to disperse the mixture.

The jars are then placed in a 38° C. water bath and left overnight.

After cooling, the samples are centrifuged at 2000 RPM in a Fisher Marathon 3200 for 15 min.

The supernatant is decanted into a weighed aluminum pan.

The insolubles are rinsed twice with 25 ml room temperature distilled water.

The rinses are centrifuged as above and combined with the supernatant.

The combined supernatant and washes are dried to steady weight at 85° C. in a forced-air oven.

The insolubles are removed and also dried in a weighed pan to steady weight at 85° C. in a forced-air oven.

The dried samples are weighed. A correction is made in the soluble portion for the weight of the buffer salts and for the weight of the enzyme added during the test.

Enzyme accessibility is calculated from this data as in the examples below. It is noted that variations in moisture content and slight variations in weighing precision can result in calculated results slightly above 100% or slightly below 0% in this method. The results shown in the following table are obtained without any correction for this type of method variance.

When the above test is run under identical conditions, but without addition of the enzyme, the test is referred to as the “Solubility Test” which is used as a control in certain examples.

In the below Enzyme Accessibility Test, an average of 95% of the untreated cellulose (cotton linters) remain insoluble. In the tables shown below, data for five replicates are presented.

| Cellulase g | 0.0613 | 0.0607 | 0.0609 | 0.0611 | 0.0610 |
|---|---|---|---|---|---|
| Cellulose (cotton | 3.22 | 3.22 | 3.22 | 3.22 | 3.22 |
| linters) g | | | | | |
| Moist. Cont. | 11.42% | 11.42% | 11.42% | 11.42% | 11.42% |
| Dry furnish g | 2.85 | 2.85 | 2.85 | 2.85 | 2.85 |
| All Solubles g | 0.71 | 0.69 | 0.69 | 0.75 | 0.70 |
| Buffer Salts + | 0.69 | 0.69 | 0.69 | 0.69 | 0.69 |
| Cellulase g | | | | | |
| Soluble Portion g | 0.02 | 0.00 | 0.00 | 0.06 | 0.01 |
| % Soluble Portion | 0.70% | 0.00% | 0.00% | 2.10% | 0.35% |
| Dry Insolubles | 2.72 | 2.72 | 2.71 | 2.69 | 2.75 |
| after washing g | | | | | |
| % Insoluble Portion | 95.36% | 95.36% | 95.01% | 94.31% | 96.41% |

| | Average | St. Dev |
|---|---|---|
| Total Solubles g | 0.71 | 0.02 |
| Buffer Salts + Cellulase g | 0.69 | 0.00 |
| Soluble Portion g | 0.02 | 0.02 |
| % Soluble Portion | 0.63% | 0.87% |
| Dry Insolubles after washing g | 2.72 | 0.02 |
| % Insoluble Portion | 95.29% | 0.76% |

In the below Enzyme Accessibility Test, cellulose treated to improve enzyme accessibility was tested. An increase in the soluble portion and a decrease in the insoluble portion was observed, when compared to the untreated cellulose controls listed in the previous table.

| Cellulase g | 0.0607 | 0.0606 | 0.0599 | 0.0604 | 0.0603 |
|---|---|---|---|---|---|
| Cellulose (cotton | 3.34 | 3.34 | 3.34 | 3.34 | 3.34 |
| linters) g | | | | | |
| Moist. Cont. | 11.60% | 11.60% | 11.60% | 11.60% | 11.60% |
| Dry furnish g | 2.95 | 2.95 | 2.95 | 2.95 | 2.95 |
| All Solubles g | 2.44 | 2.41 | 2.55 | 2.54 | 2.53 |
| Buffer Salts + | 0.63 | 0.63 | 0.63 | 0.63 | 0.63 |
| Cellulase g | | | | | |
| Soluble Portion g | 1.75 | 1.72 | 1.86 | 1.85 | 1.84 |
| % Soluble Portion | 57.21% | 56.20% | 60.97% | 60.61% | 60.28% |
| Dry Insolubles | 1.23 | 1.25 | 1.16 | 1.16 | 1.16 |
| after washing g | | | | | |
| % Insoluble Portion | 41.66% | 42.34% | 39.29% | 39.29% | 39.29% |

| | Average | St. Dev |
|---|---|---|
| Total Solubles g | 2.49 | 0.06 |
| Buffer Salts + Cellulase g | 0.63 | 0.00 |
| Soluble Portion g | 1.80 | 0.06 |
| % Soluble Portion | 61.09% | 2.19% |
| Dry Insolubles after washing g | 1.19 | 0.04 |
| % Insoluble Portion | 40.37% | 1.50% |

A polysaccharide is considered to be a disrupted polysaccharide with increased accessibility if the increase in percent soluble portion, or a decrease in the insoluble portion, as measured in a relevant Enzyme Accessibility Test, is statistically significant in comparison with its untreated polysaccharide control.

For the above-listed Enzyme Accessibility Test, the soluble portion of initial solids of the treated polysaccharide with increased accessibility was 61.09% with a standard deviation of 2.19%. The soluble portion of the control polysaccharide was 0.63% with a standard deviation of 0.87%. Therefore this treated polysaccharide was considered to be a disrupted polysaccharide with increased accessibility. Alternatively, the insoluble portions could also be compared with the same resulting conclusion.

The invention is further demonstrated by the following examples. The examples are presented to illustrate the invention, parts and percentages being by weight, unless otherwise indicated.

### EXAMPLES

### Example 1

### Disrupted Derivatized Cellulose

A disrupted cellulose was produced combining low levels of substitution, such as less than 0.4 DS, with intercalation of soluble materials such as glucose. For example, a carboxymethylcellulose (CMC) with a DS of about 0.25 made by conventional means except, with the addition of glucose during the swelling and derivatization process.

TABLE 1

| Slurry Solids | 7.59% | |
|---|---|---|
| Cellulose | 60.30 | g |
| Glucose | 6.70 | g |
| Water | 80.30 | g |
| IPA | 663.90 | g |
| NaOH (50% pure) | 71.26 | g |
| Stir 90 min. @ 5° C. | | |
| 50% MCA in IPA | 21.56 | g |

Table 1 shows a recipe wherein the ingredients in the column except for the monochloroacetic acid (MCA) solution are combined under a nitrogen blanket and allowed to stir under nitrogen for about 90 minutes at 5° C. to swell the cellulose. The 50% monochloroacetic acid in isopropanol was then combined with the alkali cellulose slurry and the mixture warmed to 70° C. to trigger the etherification. After an hour, the mixture was cooled and filtered, and the resulting fibers were neutralized in MeOH/Water (640 g/160 g) using acetic acid. After two additional washes with MeOH/Water (640 g/160 g) to remove residual salts, the material was filtered and dried on a fluid bed drier for one hour at 70° C. Unexpectedly, most of the highly soluble glucose was retained despite the aqueous methanol washes. A run without glucose gave a yield of 60.3 g when starting with 61.91 g cellulose, or 97% recovery. In the run in this example, 62.15 g were obtained after starting with 60.3 g of cellulose and 6.7 g glucose or about 103% recovery of the cellulose weight. This means that about half of the glucose was retained after washing.

### Example 2

### Galactose Disruption

In this example, galactose was used as the disrupting agent.

A commercial wood pulp, (Borregaard VHV, available from Borregaard ChemCell, Sarpsborg, Norway) was swollen in a mixture of water and ethanol and sodium hydroxide. As a control, 16.20 g wood pulp was swollen by making a slurry with 129.6 g of absolute ethanol and stirring in a mixture of 8.80 g 50% sodium hydroxide in 15.85 g distilled water. A disrupted sample was prepared as above except that 14.58 g of underivitized wood pulp was used and 1.62 g galactose was added. The following materials were used in the production of the sample: Absolute Ethanol 200 Proof (available from Spectrum Chemical Mfg. Co. Lot #YT0042), Methanol 99.8% (available from Puritan Products Lot #025118), D-(+)-Galactose (available from Sigma-Aldrich >=98%),and Sodium Hydroxide 50% in water Batch #72897MJ (available from Sigma-Aldrich).

The samples were shaken, cooled in an ice bath and left in a refrigerator at about 4° C. overnight. The liquid phase was removed by filtration, and the filter cake was slurried in 250 mls of a mixture of 200 g methanol and 50 g water. The pH of the slurry was adjusted to 7.0+/−0.1 by addition of 3.7% v/v hydrochloric acid, and 5% sodium hydroxide as needed. The samples were then filtered and washed twice with 250 g portions of 80% methanol as above. Half of each sample was used for the Enzyme Accessibility and Solubility Tests without drying, and the other half was oven dried to constant weight in a VWR 1350 FD forced air oven. Table 2A lists the results for the Solubility and Enzyme Accessibility Tests for both dried and never-dried samples.

TABLE 2A
Galactose Disruption of Wood Pulp

| | Wood Pulp VHV | VHV Wood Pulp + | VHV Wood Pulp | VHV Wood Pulp + |
|---|---|---|---|---|
| | Wood Pulp Control | 10% galactose | Control | 10% galactose |
| | Dried | | Never-dried | |
| g Insolubles without enzyme | 2.02 | 2.01 | 4.02 | 3.32 |
| (Solubility Test) | | | | |
| g Insolubles with enzyme | 1.93 | 1.86 | 3.91 | 3.00 |
| (Accessibility Test) | | | | |
| % weight loss from enzyme | 4.6% | 7.6% | 2.6% | 9.6 |
| treatment | | | | |

For both the dried and never-dried samples the addition of 10% galactose, relative to the untreated polysaccharide control, reduced the insoluble portion when evaluated using the Solubility Test (no enzyme present). The large change in insoluble fraction observed for the never dried sample showed that for that case some of the material, presumably surface adsorbed galactose, was solubilized by the test solution. The further reduction in insoluble portion, when comparing the no enzyme and enzyme tests, shows that both de-polymerization and release of the entrapped galactose contribute to the additional soluble fraction.

The soluble fractions from the Enzyme Accessibility and Solubility tests generated for the disrupted samples shown in Table 2A above were also analyzed by ion chromatography (IC). The filtrates from the wood pulp prepared with 10% galactose as a disruptor were submitted for ion chromatography analysis using high pH conditions to resolve the various sugar components. The resulting peaks were compared with standards from Sigma-Aldrich including glucose, mannose, galactose, and xylose. Concentrations (mg/g) for the various sugars present in the filtrates are shown in Table 2B

The ion chromatography analysis was performed using the following procedure and conditions. As received sample solutions were filtered at 0.45 microns and diluted to appropriate range with 10 mM NaOH and analyzed. Conditions were:

Instrument: Dionex ICS 3000

Column: Dionex PA-10 carbohydrate column

Eluent: 10 mM NaOH

Flow Rate: 1.0 mL/min

Injection: 20 uL, partial loop injection

Detector: Pulsed amperometry at a gold electrode

TABLE 2B
Galactose Recovery from Filtrates for
the Galactose Disrupted Wood Pulp

| | Data in mg sugar observed per gram | | | |
|---|---|---|---|---|
| | galactose disrupted wood pulp added | | | |
| | Galac- | Glu- | Xy- | Man- |
| | tose | cose | lose | nose |
| | Dried | | | |
| Without enzyme, Solubility Test | 1.74 | 0.05 | none | none |
| | | | detected | detected |
| With enzyme, Accessibility Test | 5.21 | 77.21 | 10.54 | 1.52 |
| Increase in obs. mg with enzyme | 3.09 | 71.4 | 10.54 | 1.52 |
| | Never-dried | | | |
| Without enzyme, Solubility Test | 10.96 | 0.03 | none | none |
| | | | detected | detected |
| With enzyme, Accessibility Test | 12.64 | 54.55 | 3.69 | 2.09 |
| Increase in obs. mg with enzyme | 1.68 | 54.52 | 3.69 | 2.09 |

For the filtrates resulting from the Solubility test (without enzyme) only galactose is observed for both the dried and never dried samples. This clearly indicates that a portion of the galactose added as a disrupting agent can be solubilized by the buffer solution used in the test.

To distinguish between galactose added as a disrupting agent and galactose present in the hemicellulose fraction of the wood starting material, a separate sugar analysis by IC was performed on the starting Borregaard VHV pulp as summarized in Table 2C. The IC sugar analysis was done as follows: 0.3 gram sample (weight to 0.001 gram) in 250 ml flask, add 3 ml of 72% H₂SO₄ for 1 hour in room temperature, stir. Add 84 ml distilled H₂O, then reflux for 5 hours. After cool down, make up to 100 ml with distilled H₂O, before analysis, dilute with 10 mM NaOH. 20 uL loads to IC. The IC condition was slightly different from that described in Table 2B. Instead using 10 mM NaOH, only 2.5 mM NaOH was used to resolve all monosaccharides. Elution time was 35 minutes. The calibration was done with all five monosaccharides standards in six concentration points and duplicate injection. All samples are the average of duplicate injections. The analysis demonstrates that only a very small amount of galactose is present in the form of hemicellulose from the starting wood pulp.

TABLE 2C
Analysis of Sugar Weight Percents in the Wood Pulp

| | Galactose | Glucose | Xylose | Mannose |
|---|---|---|---|---|
| Borregaard VHV | .08% | 89.36% | 6.17% | 4.39% |

The results from Table 2B above show, in all cases, the amount of galactose found greatly exceeds the small amounts of galactose expected from the hydrolysis of the hemicellulose component of the wood pulp, confirming that the galactose added as a disrupting agent, was retained in the treated polymer. Table 2B shows that some of the retained galactose was observed without hydrolysis by enzyme. This may simply be adsorbed on the polymer and is not removed by washing. Additional galactose was observed when the enzyme was added (see Table 2B), which is consistent with the concept that the hydrolysis-released galactose was intercalated in the cellulose polymer during the high pH swollen stage. While not wishing to be bound by theory, it is believed that this hydrolysis-released galactose made the polymer more available to the enzyme, probably through a reduction in order.

### Example 3

### Additivity of Disruptions

Hydroxyethylcelluose (HEC) and carboxymethylcellulose (CMC) were prepared with low MS and low DS, respectively. These derivatized celluloses were then reswollen and treated with disrupting agent and evaluated using the Enzyme Accessibility Test to determine the effect of the combination of etherification and sugar disruption on water solubility and on enzyme accessibility at MS or DS levels below the level that imparts water solubility to the cellulose.

Hydroxyethylcellulose (HEC) was made from a commercial wood pulp, Borregaard VHV from Borregaard ChemCell, PO box 162, Sarpsborg, Norway. The HEC was made in several runs at various low levels of molar substitution (MS) in a pilot plant using a recipe similar to that used for commercial HEC, except for the use of reduced levels of ethylene oxide to obtain reduced levels of hydroxyethylation. The products were purified by normal HEC production procedures.

The low DS CMC's were made using standard methods using Foley Fluff wood pulp, Buckeye Technologies Inc., Memphis, Tenn.

5.0 g samples of the derivatized cellulose samples were then swollen in 75.0 g 10% aqueous NaOH both in the presence of 10% by weight glucose and without glucose present and stirred in an ice bath for an hour. The samples were then kept overnight in the refrigerator. The stirred slurry was then neutralized using 17.5% hydrochloric acid to a pH of about 5.5. The samples were then filtered and washed by adding 250 g distilled water. This slurry was filtered, washed again with 250 ml water, filtered, and dried to steady weight at 85° C. in a VWR 1350FD forced-air oven.

Samples were prepared in matched pairs with and without 0.50 g cellulase enzyme. 2.5 g (corrected for moisture content) of the derivatized cellulose samples was mixed with 50.0 g pH 5.0 sodium phosphate and shaken. The remainder of the procedure is described in the Enzyme Accessibility Test. The reagents used are the same.

TABLE 3
Additivity of Enzyme Enhancement

| | MS 0.09 | MS 0.09 | DS 0.08 | DS 0.08 |
|---|---|---|---|---|
| | HEC | HEC | CMC | CMC |
| Initial polymer g | 5.00 | 5.00 | 5.00 | 5.00 |
| Glucose g | None | 0.50 | None | 0.50 |
| Drypolymer after NaOH | 4.41 | 4.73 | 4.49 | 4.56 |
| treatment and neutralization g | | | | |
| % Soluble without enzyme | 1.2 | 3.6 | 2.0 | 4.0 |
| % Soluble with enzyme | 10.5 | 32.3 | 21.8 | 25.0 |
| % Insoluble without enzyme | 88.9 | 88.5 | 87.7 | 87.3 |
| % Insoluble with enzyme | 59.8 | 56.6 | 68.7 | 64.6 |

The addition of 10% glucose to the derivatized cellulose increased the soluble portion and reduced the insoluble portion when enzyme was present. Because the increase in % soluble fraction was greater with the enzyme than without, when comparing samples with and without added glucose, it was apparent that the presence of the glucose promoted increased hydrolysis of the derivatized cellulose. Similarly, the greater decrease in insoluble portion, when comparing the case with enzyme to that without, demonstrated that de-polymerization was the main source of the additional soluble fraction, not the added glucose.

### Example 4

### Accessibility Enhancement by Addition of Substantive Disrupting Agents

In 200 ml jars, mixtures were made as shown in Table 4A.

Samples of underivatized wood pulp were first swollen in ethanol, water, NaOH mixtures both in the presence of 10% by weight disrupting agent and without disrupting agent present and shaken vigorously over ten minutes, cooled in an ice bath and left in a refrigerator at about 4° C. overnight. The liquid phase was removed by filtration, and the filter cake was slurried in 250 mls of a mixture of 200 g methanol and 50 g water. The pH of the slurry was adjusted to 7.0+/−0.1 by addition of 3.7% v/v hydrochloric acid, and 5% sodium hydroxide, as needed. The samples were then filtered and washed twice with 250 g portions of 80% methanol as described in Example 2. Half of each sample was used for the Enzyme Accessibility Test, without drying, and the other half was oven dried to constant weight in a VWR 1350 FD forced air oven.

The following materials were used in the production of the sample: Absolute Ethanol 200 Proof (available from Spectrum Chemical Mfg. Co.), Methanol 99.8% (available from Puritan Products), D-(+)-Galactose (available from Sigma-Aldrich >=98%), D(+)-Glucose (available from Sigma-Aldrich >=99%), α-D-Methyl glucose (available from Sigma-Aldrich as α-D-Methyl glucopyranoside >=99%), and Sodium Hydroxide 50% in water (available from Sigma-Aldrich).

TABLE 4A
Ingredients

| | Disrupting Agent: | | | | |
|---|---|---|---|---|---|
| | | D(+)- | | | |
| | | Glu- | α-methyl | Galac- | Cello- |
| | None | cose | glucoside | tose | biose |
| Wood Pulp g | 16.20 | 14.58 | 14.58 | 14.58 | 14.58 |
| Disrupting Agent g | 0.00 | 1.62 | 1.62 | 1.62 | 1.62 |
| 50% Sodium | 8.80 | 8.80 | 8.80 | 8.80 | 8.80 |
| Hydroxide g | | | | | |
| Distilled Water g | 15.85 | 15.85 | 15.85 | 15.85 | 15.85 |
| Absolute Ethanol g | 129.60 | 129.60 | 129.60 | 129.60 | 129.60 |

Samples were prepared in matched pairs with and without 0.50 g cellulase enzyme. 2.0 g or 4.0 g of the cellulosic was mixed with 50.0 g of pH 5.0, 50 millimolar sodium phosphate buffer and shaken. The remainder of the procedure is described in the Enzyme Accessibility Test. The reagents used were the same.

TABLE 4B
Accessibility Enhancement by Addition
of Substantive Disrupting Agents

| | Disrupting Agent: | | | | |
|---|---|---|---|---|---|
| | | D(+)- | | | |
| | | Glu- | α-methyl | Galac- | Cello- |
| | None | cose | glucoside | tose | biose |
| Dried Disrupted Cellulosic: | | | | | |
| Amount insoluble without | 2.02 | 2.02 | 2.03 | 2.01 | 1.96 |
| enzyme treatment g | | | | | |
| Amount insoluble after | 1.93 | 1.88 | 1.86 | 1.86 | 1.90 |
| enzyme treatment g | | | | | |
| % weight loss after enzyme | 4.2 | 7.5 | 8.0 | 7.5 | 2.9 |
| Undried Disrupted | | | | | |
| Cellulosic: | | | | | |
| Amount insoluble without | 4.02 | 3.33 | 3.36 | 3.32 | 3.40 |
| enzyme treatment g | | | | | |
| Amount insoluble after | 3.91 | 3.32 | 3.33 | 3.00 | 3.25 |
| enzyme treatment g | | | | | |
| % weight loss | 3.1 | 0.30 | 0.60 | 9.8 | 4.5 |

Among the dried samples, three gave significantly more weight loss than the control, when compared with and without enzyme use. For the dried samples no significant material was solubilized in the presence of the buffer when enzyme was added. This demonstrated that the disrupting agent became entrapped in the cellulose matrix when the sample was dried. For the undried samples, a significant decrease was observed in the insoluble portion without enzyme. This suggested that the process of drying entrapped a significant portion of the added disrupting agent while for the undried samples a significant amount of the disrupting agent can be solubilized and removed during Enzyme Accessibility Test. Among the undried samples, the galactose showed the greatest weight loss observed when the no enzyme and enzyme treated cases were compared.

### Example 5

### Ammonium Hydroxide Swelling Agent

Most of the Examples demonstrate the use of Sodium Hydroxide as the swelling agent, but other swelling agents may be used. In this example, a comparable strength of Ammonium Hydroxide (mole basis) was used instead. Samples were prepared along with those in Example 2.

TABLE 5A
Ingredients

| Disrupting Agent: | None | D(+)-Glucose | D(+)-Glucose |
|---|---|---|---|
| Underivitized Wood Pulp g | 16.20 | 14.58 | 14.58 |
| Disrupting Agent g | 0.00 | 1.62 | 1.62 |
| 50% Sodium Hydroxide g | 8.80 | 8.80 | 0.00 |
| Distilled Water g | 15.85 | 15.85 | 6.13 |
| Absolute Alcohol g | 129.60 | 129.60 | 0.00 |
| 190 Proof Ethanol g | 0.00 | 0.00 | 139.35 |
| Ammonium Hydroxide 30% g | 0.00 | 0.00 | 11.57 |

The Ammonium Hydroxide was from J. T. Baker, Phillipsburg N.J., Ethanol 190 Proof (non-denatured, available from J. T. Baker, Phillipsburg N.J.). The other ingredient sources were previously described.

TABLE 5B
Ammonium Hydroxide Swelling Agent

| | Disrupting Agent: | | |
|---|---|---|---|
| | | D(+)- | D(+)- |
| | | Glucose/NaOH | Glucose/Ammonium |
| | | Swelling | Hydroxide Swelling |
| | None | Agent | Agent |
| Dried Disrupted Cellulosic: | | | |
| Sample without enzyme | 2.02 | 2.02 | 2.02 |
| treatment g | | | |
| Sample after enzyme | 1.93 | 1.88 | 1.87 |
| treatment g | | | |
| % weight loss after enzyme | 4.2 | 7.5 | 7.8 |
| Undried Disrupted | | | |
| Cellulosic: | | | |
| Sample without enzyme | 4.02 | 3.33 | 3.11 |
| treatment g | | | |
| Sample after enzyme | 3.91 | 3.32 | 3.21 |
| treatment g | | | |
| % weight loss | 3.1 | 0.30 | −2.7 |

Although the undried sample did not show improvement when ammonium hydroxide was used instead of sodium hydroxide as the swelling agent, the dried sample gave an improvement comparable to the sodium hydroxide-swelled glucose sample, both nearly twice the control.

### Example 6

### Urea as a Disrupting Agent

Most of the Examples show the use of polysacharrides as the disrupting agent, but other disrupting agents may be used. In this example, urea was used. Samples were prepared by swelling 10.0 g of wood pulp (Borregaard VHV, available from Borregaard ChemCell, Sarpsborg, Norway) in 10% Sodium Hydroxide made by diluting Sodium Hydroxide 50% in water (available from Sigma-Aldrich) with distilled water.

Samples were first swollen both in the presence of 10% by weight disrupting agent and without disrupting agent present, and stirred vigorously over ten minutes, while cooling in an ice water bath and left in a refrigerator at about 4° C. overnight. The liquid phase was removed by filtration, and the filter cake was slurried in 250 mls of distilled water. The pH of the slurry was adjusted to 7.0+/−0.1 by addition of 3.7% v/v hydrochloric acid, and 5% sodium hydroxide as needed. The samples were then filtered and washed twice with 250 g portions of distilled water as above. Each sample was used for the Enzyme Accessibility Test after drying to constant weight in a VWR 1350 FD forced air oven.

TABLE 6A
Ingredients

| Disrupting Agent: | None | D(+)-Glucose | Urea |
|---|---|---|---|
| Wood Pulp g | 10.00 | 10.00 | 10.00 |
| Disrupting Agent | 0.00 | 1.00 g glucose | 1.00 g urea |
| 50% Sodium Hydroxide g | 20.00 | 20.00 | 20.00 |
| Distilled Water g | 80.00 | 80.00 | 80.00 |

The Urea was from J. T. Baker. The other ingredient sources were previously described.

TABLE 6B
Urea as a Disrupting Agent

| Disrupting Agent: | None | D(+)-Glucose | Urea |
|---|---|---|---|
| % Soluble without enzyme | 0.00 | 0.3 | 1.3 |
| % Soluble with enzyme | 10.0 | 21.5 | 19.9 |
| % Insoluble without enzyme | 97.2 | 97.5 | 96.8 |
| % Insoluble with enzyme | 83.7 | 78.9 | 79.2 |

Both the glucose control and Urea were shown to be an effective disrupting agent that withstood neutralization and subsequent washing while imparting enhanced accessibility. In both cases, no substantial solubilization was observed when enzyme was absent in the Solubility Test.

### Example 7

### A Substantive Disrupting Agent in Aqueous Media Only

In the previous examples, both alcohol/water and water only systems were used to prepare the sample during the swelling, neutralization, and washing steps. In this example, substantive disrupting agents were shown to be effective in enhancing accessibility when water was used with sodium hydroxide without alcohols or other organic solvents.

Samples were prepared as in Example 6. The formulations used are shown in Table 7A and the results from the Enzyme Accessibility and Solubility Tests are shown in Table 7B.

TABLE 7A
Ingredients

| | Disrupting Agent: | | | |
|---|---|---|---|---|
| | | 2.5% | 5% | 10% |
| | None | Galactose | Galactose | Galactose |
| Wood Pulp g | 10.00 | 10.00 | 10.00 | 10.00 |
| Disrupting Agent g | 0.00 | 0.25 | 0.50 | 1.00 |
| 50% Sodium Hydroxide g | 20.00 | 20.00 | 20.00 | 20.00 |
| Distilled Water g | 80.00 | 80.00 | 80.00 | 80.00 |

TABLE 7B
Substantive Disrupting Agent in Aqueous Media Only

| | Disrupting Agent: | | | |
|---|---|---|---|---|
| | | 2.5% | 5% | 10% |
| | None | Galactose | Galactose | Galactose |
| % Soluble without enzyme | 0.00 | 0.0 | 0.3 | 0.3 |
| % Soluble with enzyme | 10.0 | 18.6 | 14.4 | 21.5 |
| % Insoluble without enzyme | 97.2 | 97.8 | 98.1 | 97.5 |
| % Insoluble with enzyme | 83.7 | 79.8 | 79.5 | 78.9 |

Although the soluble portion of the 5% galactose sample was somewhat lower than the other samples in this Example, each of the treated samples was substantially higher in soluble portion than the control. Each of the samples showed a reduced % insoluble portion when compared to the no enzyme and enzyme cases, suggesting a significant enhancement in enzymatic hydrolysis of the cellulose because of the action of the disrupting agent. The data showed that as little as 2.5% added disrupting agent can be effective.

### Example 8

### Disruption of Cellulose Using Glucose

This example is similar to Example 2, except that glucose was used as the disrupting agent.

An underivatized wood pulp, (Borregaard VHV, available from Borregaard ChemCell, Sarpsborg, Norway) was swollen in a mixture of ethanol, water and sodium hydroxide. As a control, 16.20 g wood pulp was swollen by making a slurry with 129.6 g of absolute ethanol and stirring in a mixture of 8.80 g 50% sodium hydroxide in 15.85 g distilled water. A disrupted sample was prepared as above except that 14.58 g of underivitized wood pulp was used and 1.62 g glucose was added. The following materials were used in the production of the sample: Absolute Ethanol 200 Proof (available from Spectrum Chemical Mfg. Co.), Methanol 99.8% (available from Puritan Products), D-(+)-Glucose (available from Acros, Reagent Grade), and Sodium Hydroxide 50% in water (available from Sigma-Aldrich).

The samples were shaken, cooled in an ice bath and left in a refrigerator at about 4° C. overnight. The liquid phase was removed by filtration, and the filter cake was slurried in 250 mls of a mixture of 200 g methanol and 50 g water. The pH of the slurry was adjusted to 7.0+/−0.1 by addition of 3.7% v/v hydrochloric acid, and 5% sodium hydroxide as needed. The samples were then filtered and washed twice with 250 g portions of 80% methanol as above. Half of each sample was used for the Enzyme Accessibility and Solubility Tests without drying, and the other half was oven dried to constant weight in a VWR 1350 FD forced air oven.

TABLE 8A
Glucose Disruption of Wood Pulp

| | VHV Wood Pulp | VHV Wood Pulp + | VHV Wood Pulp | VHV Wood Pulp + |
|---|---|---|---|---|
| | Control | 10% glucose | Control | 10% glucose |
| | Dried | | Never-dried | |
| Amount Insolubles without enzyme | 2.02 | 2.02 | 4.02 | 3.33 |
| (Solubility Test) g | | | | |
| Amount Insolubles with enzyme | 1.93 | 1.88 | 3.91 | 3.32 |
| (Enzyme Accessibility Test) g | | | | |
| % weight loss from enzyme treatment | 4.6% | 7.2% | 2.6 | 0.3% |

The addition of 10% glucose relative to the untreated polysaccharide reduced the insoluble portion when no enzyme was present compared with comparable samples prepared without glucose. The large change in insoluble fraction observed for the never dried sample showed that for that case some of the material, presumably surface adsorbed glucose, was solubilized by the test solution. The further reduction in insoluble portion for the dried sample, when the no enzyme and enzyme tests were compared, showed that both depolymerization and release of the entrapped glucose contributed to the additional soluble fraction.

The soluble fractions from the Enzyme Accessibility and Solubility Tests generated in Example 8 above were also analyzed by ion chromatography. The filtrates from the wood pulp prepared with 10% glucose as a disruptor were submitted for ion chromatography analysis using high pH conditions to resolve the various sugar components. The resulting peaks were compared with a glucose standard from Sigma-Aldrich. Results are summarized in Table 8B.

The ion chromatography analysis was performed using the following procedure and conditions. As received sample solutions were filtered at 0.45 microns and diluted to appropriate range with 10 mM NaOH and analyzed. Conditions were:

Instrument: Dionex ICS 3000

Column: Dionex PA-10 carbohydrate column

Eluent: 10 mM NaOH

Flow Rate: 1.0 mL/min

Injection: 20 uL, partial loop injection

Detector: Pulsed amperometry at a gold electrode

TABLE 8B
Glucose Recovery from Filtrates for
the Glucose Disrupted Wood Pulp

| | Data in ppm sugar observed per gram | | |
|---|---|---|---|
| | glucose disrupted wood pulp initial | | |
| | Control | 10% | % Increase in |
| | (no glucose | Glu- | glucose ppm |
| | added) | cose | with enzyme |
| Dried | | | |
| Without enzyme, Solubility Test | <10 | <10 | |
| With enzyme, Enzyme | 3261 | 3,511 | 7.7% |
| Accessibility Test | | | |
| Never dried | | | |
| Without enzyme, Solubility Test | <10 | <10 | |
| With enzyme, Enzyme | 300 | 16,847 | 5615.66% |
| Accessibility Test | | | |

Little or no glucose was detected in this test without enzyme, and with enzyme an increase was seen for the case of the dried treated pulp compared to the control. In the case of the never-dried sample a very large increase was seen, suggesting that the dried sample may have hornified upon drying, thus decreasing enzyme availability relative to the never-dried sample.

Because the samples in this example were disrupted using the same sugar as is produced by the enzyme hydrolysis of cellulose, it is difficult to prove that enzyme accessibility is enhanced from this data alone. By comparing these results with those in Example 2, which used a different sugar as the disrupter, it may be seen that in each case disruption enhanced hydrolysis yield, as measured both by weight loss of insolubles and by increased glucose yields in the filtrates.

It is not intended that the examples given here should be construed to limit the invention, but rather they are submitted to illustrate some of the specific embodiments of the invention. Various modifications and variations of the present invention can be made without departing from the scope of the appended claims.

We claim:

1. A process for producing fermentable sugars derivable from a biomass that contains polysaccharide comprising the steps of:
obtaining the biomass;
treating the biomass with a swelling agent and;
contacting the biomass with a disrupting agent to produce a polysaccharide with increased accessibility; and
converting the polysaccharide with increased accessibility to fermentable sugars by hydrolysis, wherein the polysaccharide with increased accessibility exhibits an increase in its soluble portion from its initial solids as determined by a relevant Enzyme Accessibility Test.

2. The process of claim 1, further comprising the step of removal or neutralization of the swelling agent after the biomass is contacted with the disrupting agent.

3. The process of claim 1, wherein the disrupting agent is substantive to or becomes entrapped within the polysaccharide.

4. The process of claim 1, wherein the disrupting agent is selected from the group consisting of fermentable sugars, nonfermentable sugars, hydroxyl or lactone containing molecules derived from sugar degradation, urea, amines and polyols.

5. The process of claim 3, wherein the disrupting agent has a molecular weight between about 60 to about 400 Daltons.

6. The process of claim 1, wherein the disrupting agent is selected from the group consisting of organic molecules containing hydroxyl groups, lactones, and water soluble ethers.

7. The process of claim 1, wherein the disrupting agent is selected from the group consisting of amines, amino acids, sulfates, and phosphates.

8. The process of claim 4, wherein the disrupting agent comprises a fermentable sugar.

9. The process of claim 8, wherein the polysaccharide comprises cellulose and the fermentable sugar comprises glucose.

10. The process of claim 1, wherein the hydrolysis of the polysaccharide with increased accessibility, further comprises the step of contacting the polysaccharide with increased accessibility, with a saccharification enzyme or enzymes under suitable conditions to produce fermentable sugars.

11. The process of claim 1, wherein the hydrolysis of the polysaccharide with increased accessibility further comprises the step of acid hydrolysis of the polysaccharide with increased accessibility to produce fermentable sugars.

12. The process of claim 1, wherein the polysaccharide is selected from the group consisting of cellulose, derivatized cellulose, hemicellulose, chitin, chitosan, guar gum, pectin, alginate, agar, xanthan, starch, amylose, amylopectin, alternan, gellan, mutan, dextran, pullulan, fructan, locust bean gum, carrageenan, glycogen, glycosaminoglycans, murein, and bacterial capsular polysaccharides.

13. The process of claim 1, wherein the biomass is selected from the group consisting of corn grain, corn cobs, crop residues such as corn husks, corn stover, cotton, cotton linters, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, wood pulp, shrubs and bushes, vegetables, fruits, flowers, animal manure, bacteria, algae and fungi.

14. The process of claim 12, wherein the polysaccharide comprises cellulose.

15. The process of claim 14, wherein the cellulose comprises a derivatized cellulose.

16. The process of claim 15, wherein the derivatized cellulose is selected from the group consisting of hydroxyethyl cellulose, ethylhydroxyethyl cellulose, carboxymethylcellulose, carboxymethylhydroxyethyl cellulose, hydroxypropylhydroxyethyl cellulose, methylcellulose, ethylcellulose, methylhydroxypropyl cellulose, methylhydroxyethyl cellulose, carboxymethylmethyl cellulose, hydrophobically modified carboxymethyl cellulose, hydrophobically modified hydroxyethyl cellulose, hydrophobically modified hydroxypropyl cellulose, hydrophobically modified ethylhydroxyethyl cellulose, hydrophobically modified carboxymethylhydroxyethyl cellulose, hydrophobically modified hydroxypropylhydroxyethyl cellulose, hydrophobically modified methyl cellulose, hydrophobically modified methylhydroxypropyl cellulose, hydrophobically modified methylhydroxyethyl cellulose, hydrophobically modified carboxymethylmethyl cellulose, nitrocellulose, cellulose acetate, cellulose sulfate, cellulose vinyl sulfate, cellulose phosphate, methylol cellulose, and cellulose phosphonate.

17. The process of claim 16, wherein the derivatized cellulose is carboxymethylcellulose.

18. The process of claim 16, wherein the derivatized cellulose is hydroxyethylcellulose.

19. The process of claim 1, wherein the swelling agent is selected from the group consisting of alkali metal oxides, alkali metal hydroxides, alkaline earth metal oxides, alkaline earth metal hydroxides, alkali silicates, alkali aluminates, alkali carbonates, amines, ammonia, ammonium hydroxide; tetramethyl ammonium hydroxide; lithium chloride; N-methyl morpholine N-oxide, urea and mixtures thereof.

20. The process of claim 19, wherein the swelling agent comprises sodium hydroxide.

21. The process of claim 19, wherein the swelling agent comprises ammonium hydroxide.

22. The process of claim 2, further comprising the step of drying the polysaccharide with increased accessibility.

23. The process of claim 2, wherein the disrupting agent is incorporated and retained within the polysaccharide with increased accessibility.

24. The process of claim 1, further comprising the step of feeding back a portion of the fermentable sugar chemical back into the process, to contact the biomass with fermentable sugar as the disrupting agent producing a polysaccharide with increased accessibility for subsequent conversion to fermentable sugars by hydrolysis.

25. A process for producing a target chemical derivable from biomass containing polysaccharide comprising the steps of:
obtaining a biomass that contains polysaccharide;
treating the biomass with a swelling agent and;
contacting the biomass that contains polysaccharide with a disrupting agent producing a polysaccharide with increased accessibility;
converting the polysaccharide with increased accessibility to fermentable sugars by hydrolysis under suitable conditions to produce fermentable sugars; and
contacting the fermentable sugars with at least one biocatalyst able to ferment the fermentable sugars to produce a target chemical under suitable fermentation conditions, wherein the polysaccharide with increased accessibility exhibits an increase in its soluble portion of initial solids as determined by a relevant Enzyme Accessibility Test.

26. The process of claim 25 wherein the target chemical is selected from the group consisting of alcohols, aldehydes, ketones and acids.

27. The process of claim 26 wherein the target chemical comprises alcohol.

28. The process of claim 27, wherein the alcohol comprises ethanol.

29. The process of claim 27, wherein the alcohol comprises butanol.

30. The process of claim 25, further comprising the step of removal or neutralization of the swelling agent after the biomass is contacted with the disrupting agent.

31. The process of claim 25, wherein the disrupting agent is selected from the group consisting of fermentable sugars, nonfermentable sugars, urea, amines, and low molecular weight polyethylene glycols.

32. The process of claim 31, wherein the disrupting agent comprises a fermentable sugar.

33. The process of claim 32, wherein the polysaccharide comprises cellulose and the fermentable sugar comprises glucose.

34. The process of claim 25, wherein the polysaccharide is selected from the group consisting of cellulose, derivatized cellulose, hemicellulose, chitin, chitosan, guar gum, pectin, alginate, agar, xanthan, starch, amylose, amylopectin, alternan, gellan, mutan, dextran, pullulan, fructan, locust bean gum, carrageenan, glycogen, glycosaminoglycans, murein, and bacterial capsular polysaccharides.

35. The process of claim 25, wherein the biomass is selected from the group consisting of corn grain, corn cobs, crop residues such as corn husks, corn stover, cotton, cotton linters, grasses, wheat, wheat straw, barley, barley straw, hay, rice straw, switchgrass, waste paper or post consumer paper, sugar cane bagasse, sorghum, soy, components obtained from milling of grains, trees, branches, roots, leaves, wood chips, sawdust, wood pulp, shrubs and bushes, vegetables, fruits, flowers, animal manure, bacteria, algae and fungi.

36. The process of claim 34, wherein the polysaccharide comprises cellulose.

37. The process of claim 34, wherein the polysaccharide comprises derivatized cellulose.

38. The process of claim 37, wherein the derivatized cellulose comprises carboxymethylcellulose.

39. The process of claim 37, wherein the derivatized cellulose comprises hydroxyethylcellulose.

40. The process of claim 25, wherein the swelling agent is selected from the group consisting of alkali metal oxides, alkali metal hydroxides, alkaline earth metal oxides, alkaline earth metal hydroxides, alkali silicates, alkali aluminates, alkali carbonates, amines, ammonia, ammonium hydroxide; tetramethyl ammonium hydroxide; lithium chloride; N-methyl morpholine N-oxide, urea and mixtures thereof.

41. The process of claim 25, wherein the swelling agent comprises sodium hydroxide.

42. The process of claim 25, wherein the swelling agent comprises ammonium hydroxide.

43. The process of claim 25, wherein the hydrolysis of the polysaccharide with increased accessibility further comprising the step of contacting the polysaccharide with increased accessibility with a saccharification enzyme or enzymes under suitable conditions to produce fermentable sugars.

44. The process of claim 25 further comprising the step of feeding back a portion of the fermentable sugar chemical back into the process, to contact the biomass with fermentable sugar as the disrupting agent producing a polysaccharide with increased accessibility for subsequent conversion to fermentable sugars by hydrolysis.

45. A process for producing a polysaccharide with increased accessibility comprising the steps of:
obtaining the polysaccharide;
treating the polysaccharide with a swelling agent and;
contacting the polysaccharide with a disrupting agent to produce a polysaccharide with increased accessibility, wherein the polysaccharide with increased accessibility exhibits an increase in its soluble portion from its initial solids as determined by a relevant Enzyme Accessibility Test.

46. The process of claim 45, further comprising the step of removal or neutralization of the swelling agent after the polysaccharide is contacted with the disrupting agent.

47. The process of claim 45, wherein the disrupting agent is substantive to the polysaccharide.

48. The process of claim 47, wherein the disrupting agent is selected from the group consisting of fermentable sugars, nonfermentable sugars, hydroxyl or lactone containing molecules derived from sugar degradation, urea, amines, and polyols.

49. The process of claim 45, wherein the disrupting agent has a molecular weight between about 60 to about 400 Daltons.

50. The process of claim 49, wherein the disrupting agent is selected from the group consisting of organic molecules containing hydroxyl groups, lactones, and water soluble ethers.

51. The process of claim 49, wherein the disrupting agent is selected from the group consisting of amines, amino acids, sulfates, and phosphates.

52. The process of claim 48, wherein the disrupting agent comprises a fermentable sugar.

53. The process of claim 52, wherein the polysaccharide comprises cellulose and the fermentable sugar comprises glucose.

54. The process of claim 45, wherein the polysaccharide is selected from the group consisting of cellulose, derivatized cellulose, hemicellulose, chitin, chitosan, guar gum, pectin, alginate, agar, xanthan, starch, amylose, amylopectin, alternan, gellan, mutan, dextran, pullulan, fructan, locust bean gum, carrageenan, glycogen, glycosaminoglycans, murein, and bacterial capsular polysaccharides.

55. The process of claim 54, wherein the polysaccharide comprises cellulose.

56. The process of claim 55, wherein the cellulose comprises a derivatized cellulose.

57. The process of claim 56, wherein the derivatized cellulose is selected from the group consisting of hydroxyethyl cellulose, ethylhydroxyethyl cellulose, carboxymethylcellulose, carboxymethylhydroxyethyl cellulose, hydroxypropylhydroxyethyl cellulose, methylcellulose, ethylcellulose, methylhydroxypropyl cellulose, methylhydroxyethyl cellulose, carboxymethylmethyl cellulose, hydrophobically modified carboxymethyl cellulose, hydrophobically modified hydroxyethyl cellulose, hydrophobically modified hydroxypropyl cellulose, hydrophobically modified ethylhydroxyethyl cellulose, hydrophobically modified carboxymethylhydroxyethyl cellulose, hydrophobically modified hydroxypropylhydroxyethyl cellulose, hydrophobically modified methyl cellulose, hydrophobically modified methylhydroxypropyl cellulose, hydrophobically modified methylhydroxyethyl cellulose, hydrophobically modified carboxymethylmethyl cellulose, nitrocellulose, cellulose acetate, cellulose sulfate, cellulose vinyl sulfate, cellulose phosphate, methylol cellulose, and cellulose phosphonate.

58. The process of claim 57, wherein the derivatized cellulose is carboxymethylcellulose.

59. The process of claim 57, wherein the derivatized cellulose is hydroxyethylcellulose.

60. The process of claim 45, wherein the swelling agent is selected from the group consisting of alkali metal oxides, alkali metal hydroxides, alkaline earth metal oxides, alkaline earth metal hydroxides, alkali silicates, alkali aluminates, alkali carbonates, amines, ammonia, ammonium hydroxide; tetramethyl ammonium hydroxide; lithium chloride; N-methyl morpholine N-oxide, urea and mixtures thereof.

61. The process of claim 60, wherein the swelling agent comprises sodium hydroxide.

62. The process of claim 60, wherein the swelling agent comprises ammonium hydroxide.

63. The process of claim 45, further comprising the step of drying the polysaccharide with increased accessibility.

64. The process of claim 63, wherein the disrupting agent is incorporated and retained within the polysaccharide with increased accessibility.

65. A polysaccharide with increased accessibility comprising
a polysaccharide, and
a disrupting agent,
wherein the disrupting agent is physically adsorbed onto, substantive to, or entrapped in the polysaccharide with increased accessibility and wherein the polysaccharide with increased accessibility exhibits an increase in its soluble portion from its initial solids as determined by a relevant Enzyme Accessibility Test.

66. The polysaccharide with increased accessibility of claim 65, wherein the disrupting agent is selected from the group consisting of fermentable sugars, nonfermentable sugars, hydroxyl or lactone containing molecules derived from sugar degradation, urea, amines, and polyols.

67. The polysaccharide with increased accessibility of claim 65, wherein the disrupting agent has a molecular weight between about 60 to about 400 Daltons.

68. The polysaccharide with increased accessibility of claim 65, wherein the disrupting agent is selected from the group consisting of organic molecules containing hydroxyl groups, lactones, and water soluble ethers.

69. The polysaccharide with increased accessibility of claim 65, wherein the disrupting agent is selected from the group consisting of amines, amino acids, sulfates, and phosphates.

70. The polysaccharide with increased accessibility of claim 66, wherein the disrupting agent comprises a fermentable sugar.

71. The polysaccharide with increased accessibility of claim 70, wherein the polysaccharide comprises cellulose and the fermentable sugar comprises glucose.

72. The polysaccharide with increased accessibility of claim 65, wherein the polysaccharide is selected from the group consisting of cellulose, derivatized cellulose, hemicellulose, chitin, chitosan, guar gum, pectin, alginate, agar, xanthan, starch, amylose, amylopectin, alternan, gellan, mutan, dextran, pullulan, fructan, locust bean gum, carrageenan, glycogen, glycosaminoglycans, murein, and bacterial capsular polysaccharides.

73. The polysaccharide with increased accessibility of claim 72, wherein the polysaccharide comprises cellulose.

74. The polysaccharide with increased accessibility of claim 73, wherein the cellulose comprises a derivatized cellulose.

75. The polysaccharide with increased accessibility of claim 74, wherein the derivatized cellulose is selected from the group consisting of hydroxyethyl cellulose, ethylhydroxyethyl cellulose, carboxymethylcellulose, carboxymethylhydroxyethyl cellulose, hydroxypropylhydroxyethyl cellulose, methyl cellulose, ethylcellulose, methylhydroxypropyl cellulose, methylhydroxyethyl cellulose, carboxymethylmethyl cellulose, hydrophobically modified carboxymethyl cellulose, hydrophobically modified hydroxyethyl cellulose, hydrophobically modified hydroxypropyl cellulose, hydrophobically modified ethylhydroxyethyl cellulose, hydrophobically modified carboxymethylhydroxyethyl cellulose, hydrophobically modified hydroxypropylhydroxyethyl cellulose, hydrophobically modified methyl cellulose, hydrophobically modified methylhydroxypropyl cellulose, hydrophobically modified methylhydroxyethyl cellulose, hydrophobically modified carboxymethylmethyl cellulose, nitrocellulose, cellulose acetate, cellulose sulfate, cellulose vinyl sulfate, cellulose phosphate, methylol cellulose, and cellulose phosphonate.

76. The polysaccharide with increased accessibility of claim 75, wherein the derivatized cellulose is carboxymethylcellulose.

77. The polysaccharide with increased accessibility of claim 75, wherein the derivatized cellulose is hydroxyethylcellulose.

78. The polysaccharide with increased accessibility of claim 75, wherein the derivatized cellulose is methylcellulose.

79. The polysaccharide with increased accessibility of claim 75, wherein the derivatized cellulose is ethylcellulose.

80. The polysaccharide with increased accessibility of claim 73, wherein the disrupting agent is selected from the group consisting of fermentable sugars, nonfermentable sugars, hydroxyl or lactone containing molecules derived from sugar degradation, urea, amines, and polyols.

81. The polysaccharide with increased accessibility of claim 80, wherein the fermentable sugar comprises glucose.

82. The polysaccharide with increased accessibility of claim 74, wherein the disrupting agent comprises a fermentable sugar.

83. The polysaccharide with increased accessibility of claim 82, wherein the disrupting agent comprises a fermentable sugar.

84. The polysaccharide with increased accessibility of claim 76, wherein the disrupting agent comprises glucose.

85. The polysaccharide with increased accessibility of claim 77, wherein the disrupting agent comprises glucose.

86. The polysaccharide with increased accessibility of claim 65, wherein the disrupting agent is substantive to the polysaccharide.
